SEQUENCE LISTING (15 sequences)

SEQ ID NO: 1 - skipped

SEQ ID NO: 2 - protein, 5 aa, synthetic construct
VSRKR

SEQ ID NO: 3 - protein, 5 aa, synthetic construct
VSKRR

SEQ ID NO: 4 - protein, 5 aa, synthetic construct
VSRRR

SEQ ID NO: 5 - protein, 5 aa, synthetic construct
VARKL

SEQ ID NO: 6 - protein, 5 aa, synthetic construct
RHKKL

SEQ ID NO: 7 - protein, 5 aa, synthetic construct
RARRL

SEQ ID NO: 8 - protein, 5 aa, synthetic construct
RSKKL

SEQ ID NO: 9 - protein, 5 aa, synthetic construct
RHKRR

SEQ ID NO: 10 - protein, 5 aa, synthetic construct
VARRL

SEQ ID NO: 11 - protein, 5 aa, synthetic construct
VARRK

SEQ ID NO: 12 - protein, 5 aa, synthetic construct
RSKRR

SEQ ID NO: 13 - skipped

SEQ ID NO: 14 - protein, 5 aa, synthetic construct
VSKKR

SEQ ID NO: 15 - protein, 12 aa, synthetic construct
ASHLRKLRKRLL

(19) United States
(12) Patent Application Publication
(10) Pub. No.: US 2024/0350578 A1
(43) Pub. Date: Oct. 24, 2024

(54) FUNCTIONAL OUTCOMES AFTER SPINAL CORD INJURY

(71) Applicant: Duke University, Durham, NC (US)

(72) Inventors: Daniel T. Laskowitz, Chapel Hill, NC (US); Timothy D. Faw, Durham, NC (US)

(21) Appl. No.: 18/639,337

(22) Filed: Apr. 18, 2024

Related U.S. Application Data
(60) Provisional application No. 63/497,096, filed on Apr. 19, 2023.

(51) Int. Cl. A61K 38/08; A61K 9/00; A61P 25/00

(52) U.S. Cl. CPC A61K 38/08; A61K 9/0019; A61P 25/00

(57) ABSTRACT

Provided herein is a method for improving functional outcomes of a spinal cord injury in a subject in need thereof, comprising administering to said subject an effective amount a peptide of Formula I, or a pharmaceutically acceptable salt thereof. Such functional outcomes may include, but are not limited to, motor, sensory, and autonomic physiological functions.

### CROSS REFERENCE TO RELATED APPLICATIONS

This application claims the benefit of and priority to U.S. Provisional Patent Application Ser. No. 63/497,096 filed Apr. 19, 2023, the disclosure of which is incorporated herein by reference in its entirety.

### STATEMENT REGARDING ELECTRONIC FILING OF A SEQUENCE LISTING

A Sequence Listing in XML text format, entitled 1175-11_ST26.xml, 12,691 bytes in size, generated on Apr. 4, 2024, and filed herewith, is hereby incorporated by reference into the specification for its disclosures.

### BACKGROUND

Spinal cord injury (SCI) affects more than 27 million people worldwide (James et al., 2019) and is the second leading cause of paralysis in the United States (Armour et al., 2016). While the scientific understanding of SCI and recovery continues to grow, a cure for the vast motor, sensory, and autonomic deficits remains elusive for the approximately 290,000 people living with SCI throughout the United States (NSCISC, 2019). There is a critical unmet need for therapeutics that reduce damage and improve mobility and bladder function after SCI, as gains in these areas improve individuals' quality of life (Goulet et al., 2019; Kachourbos & Creasey, 2000) and reduce both rehospitalization (DeJong et al., 2013) and overall lifetime costs (Morrison et al., 2018). Therefore, development of a therapeutic that could be administered at the point of care or acutely after SCI to protect the spinal cord from chronic effects of trauma would be a significant asset to the general population.

One promising, but understudied, area is the role of apolipoprotein E (APOE: gene; apoE: protein), the major apolipoprotein produced in the central nervous system (CNS), in modulating neuroinflammation and neuroplasticity after SCI. Originally described in the context of cholesterol transport, the 299-amino acid protein has numerous biological functions both outside and within the CNS (Mahley, 1988). Indeed, APOE genotype, of which there are three major isoforms that differ by single amino acid interchanges at positions 112 and 158 (Weisgraber, 1994), has emerged as an important genetic modifier of patient outcomes after CNS injury. Carriers of the APOE4 allele demonstrate worse clinical outcomes following cerebrovascular disease (James et al., 2009; Lanterna et al., 2007), traumatic brain injury (TBI) (Lawrence et al., 2015; Zhou et al., 2008), or SCI (Jha et al., 2008; Sun et al., 2011). Endogenous apoE, produced in the CNS primarily by astrocytes, is upregulated following CNS injury, where it modifies recovery from neurotrauma in a multifunctional manner by downregulating glial release of proinflammatory mediators (Aono et al., 2003; Laskowitz, Goel, et al., 1997; Laskowitz et al., 2001) and promoting neurite outgrowth (Boyles et al., 1985; Nathan et al., 2005; Nathan, 1994; Seitz et al., 2003). With the exception of apoE produced by infiltrating macrophages and neutrophils (Seitz et al., 2003), the CNS pool of apoE is largely sequestered from peripheral sources as the intact holoprotein is too large to cross the blood-brain or blood-spinal cord barrier (BSCB) (Linton et al., 1991).

Endogenous apoE in the CNS modulates neuroinflammatory responses and outcomes after brain injury (Laskowitz, Goel, et al., 1997; Laskowitz et al., 1998), and preclinical studies have confirmed that reduction of apoE activity in the CNS via genetic knock-out is associated with increased tissue injury and worse functional outcomes after CNS trauma, including SCI (Cheng et al., 2018; Laskowitz, Sheng, et al., 1997; Sheng et al., 1999; Yang et al., 2018). Following neuronal injury, apoE is upregulated in the CNS where it binds to cell surface receptors on microglia, which are the resident immune cells of the CNS and are among the first cells to respond to damage (Donnelly & Popovich, 2008; Hansen et al., 2013; Rice et al., 2007). Early after SCI, activated microglia participate in scar formation, phagocytosis, and clearing of myelin debris at the site of primary damage (Bellver-Landete et al., 2019; Brennan et al., 2018; Greenhalgh & David, 2014). However, they also express pro-inflammatory cytokines, tumor necrosis factor alpha (TNFα) and interleukin-1 beta (IL-1β), as well as reactive oxygen species and chemokines, such as C—C motif chemokine ligand 2 (CCL2) (Fleming et al., 2006; Norden et al., 2019). The inflammatory microenvironment created in part by microglia ultimately leads to trafficking of peripheral immune cells, further BSCB breakdown, and ultimately progression of secondary tissue damage that exacerbates the initial trauma (Brennan et al., 2018; Carlson et al., 1998; Town et al., 2005).

In addition to microglia, it has been shown that peripheral immune cells, such as macrophages, infiltrate the injured spinal cord, where they play diverse roles that include contributing to neuroinflammation and secondary pathology (Evans et al., 2014; Greenhalgh & David, 2014; Hansen et al., 2016; Kigerl et al., 2009; Norden et al., 2019; Popovich & Hickey, 2001; Zhu et al., 2015). Indeed, hematogenous macrophages are observed in the lesion site as early as 24 hours post injury, reach peak numbers between three- and seven-days post-injury, and remain indefinitely (Kigerl et al., 2009; Popovich & Hickey, 2001; Popovich et al., 1997). Macrophages in the injury site have diverse functions that can be beneficial, deleterious, or both simultaneously (Gensel et al., 2009). Beginning three days post injury, hematogenous macrophages begin to take over the brunt of the phagocytosis burden from the resident microglia population (Greenhalgh & David, 2014). The hematogenous macrophage population fills the lesion cavity where most take on a pro-inflammatory phenotype (Kigerl et al., 2009; Popovich & Hickey, 2001), expressing pro-inflammatory markers such as inducible nitric oxide synthase, cluster of differentiation (CD)-16, CD-86, CD-64, and CD-32 (Kigerl et al., 2009). They also contribute to the fibrotic scar formation and ultimately inhibit neurite outgrowth at the epicenter (Zhu et al., 2015). Additionally, hematogenous macrophages contribute to axon dieback over the first week following SCI, beginning at two days post injury (Evans et al., 2014). This response is significant as greater axon sparing is correlated with reduced microglial activation, BSCB permeability, and improved locomotor performance (Basso et al., 1996; Basso et al., 2006; Ma et al., 2001; Popovich et al., 2002; Popovich et al., 1996). Importantly, the absence of microglia results in lesion expansion and worsened functional outcomes after SCI (Bellver-Landete et al., 2019; Brennan et al., 2018), while preventing macrophage responses has shown conflicting results (Greenhalgh et al., 2018; Popovich et al., 1999; Zhu et al., 2015).

As such, therapeutic approaches that target the neuroinflammatory response must seek to modulate these cellular responses rather than to eliminate them. Interestingly, binding of apoE to the low-density lipoprotein receptor (LRP-1) on microglia or macrophages ultimately reduces their natural pro-inflammatory response to injury (Pocivavsek et al., 2009; Yang et al., 2016; Zurhove et al., 2008), thus altering the trajectory post-injury inflammatory cascades and resulting secondary tissue damage. This is especially important in the context of SCI, as both neuroinflammatory responses and secondary tissue injury are significantly greater after SCI than following other traumatic CNS injuries (Batchelor et al., 2008; Schnell et al., 1999).

Modulating microglial and macrophage responses through apoE has shown potential to protect against secondary injury by reducing glial activation and release of inflammatory mediators and neuronal susceptibility to excitotoxicity and oxidative stress (Aono et al., 2003; Laskowitz, Goel, et al., 1997; Laskowitz et al., 2001). However, pharmacologically augmenting endogenous CNS apoE through peripheral injection is ineffective given the size and inability of the intact apoE protein to cross the blood-CNS barriers (Linton et al., 1991). To address this limitation, we created a series of apoE-mimetic peptides derived from the receptor-binding region of the apoE protein (residues 130-149), many of which (apoE(133-149), COG112, COG1410) retained the anti-inflammatory and neuroprotective properties of the native holoprotein (Laskowitz et al., 2001). Moreover, we found that these apoE-mimetic peptides bound and initiated signal transduction through the LRP-1 receptor found on microglia and macrophages (Misra et al., 2001). Additionally, and perhaps most importantly, these peptides were able to cross the blood-brain barrier to reduce both systemic and CNS inflammatory responses following lipopolysaccharide (LPS) injection (Lynch et al., 2003) as well as reduce glial activation, release of pro-inflammatory cytokines, and improve both functional and histological outcomes in preclinical models of brain injury (Laskowitz et al., 2012; Laskowitz et al., 2007; Laskowitz et al., 2010; Wang et al., 2013).

While decades of research have been dedicated to understanding the role of apoE in the brain and the development of apoE-mimetic peptides to improve recovery after brain injury, the SCI field has only recently begun to take note. Consistent with the brain injury literature, at least two studies show a relationship between APOE genotype and functional outcomes in clinical populations with SCI. Indeed, individuals carrying the APOE4 allele had longer rehabilitation stays and worse motor and pain sensation outcomes on the American Spinal Injury Association International Standards for Classification of SCI exam than those without the APOE4 allele (Jha et al., 2008; Sun et al., 2011). Further, preclinical studies confirm that reducing apoE activity via genetic knock-out worsens tissue damage and functional outcomes after SCI (Cheng et al., 2018; Yang et al., 2018). To date, three studies have investigated the ability of early apoE-mimetic peptides, derived from the receptor-binding region of the apoE protein, to improve neuropathology and recovery in preclinical SCI models (Cheng et al., 2018; Gu et al., 2013; Wang et al., 2014).

The first of these experiments showed reduced demyelination, axon preservation, and improved locomotor recovery when an apoE-mimetic-peptide, COG112, was delivered immediately following lysolecithin demyelination in the spinal cord (Gu et al., 2013). The next showed that treatment with apoE-mimetic peptide, COG1410, beginning five minutes after a contusive SCI reduced microgliosis, lesion size, and improved locomotion 14 days after injury in a preclinical rat model (Wang et al., 2014). Most recently, Cheng and colleagues demonstrated that COG112, delivered beginning 30 minutes post-injury, improved BSCB integrity, reduced leukocyte infiltration, increased tissue sparing, and improved locomotor function in mice with a moderate, contusive SCI (Cheng et al., 2018).

Interestingly, none of these preclinical studies investigated the effect of apoE-mimetic peptides on sensory function, despite the association of APOE with pain outcomes in clinical populations.

### SUMMARY

Provided herein according to some embodiments is a method for improving one or more functional outcomes of a spinal cord injury in a subject in need thereof, comprising administering to said subject an effective amount a peptide of Formula I: X1-X2-X3-X4-X5 (SEQ ID NO:1) or a salt thereof, wherein X1 is V or R; X2 is S, A, or H; X3 is K or R; X4 is K or R; and X5 is R, L, or K, or a pharmaceutically acceptable salt thereof. In some embodiments, the peptide is N-terminal acetylated and/or C-terminal amidated.

In some embodiments, the peptide of Formula 1 is VSRKR (SEQ ID NO:2), VSKRR (SEQ ID NO:3), VSRRR (SEQ ID NO:4), VARKL (SEQ ID NO:5), RHKKL (SEQ ID NO:6), RARRL (SEQ ID NO:7), RSKKL (SEQ ID NO:8), RHKRR (SEQ ID NO:9), VARRL (SEQ ID NO:10), VARRK (SEQ ID NO:11), or RSKRR (SEQ ID NO:12), or a pharmaceutically acceptable salt thereof.

In some embodiments, the peptide is provided in a pharmaceutically acceptable carrier. In some embodiments, the pharmaceutically acceptable carrier is suitable for administering said peptide by injection or infusion (e.g., sterile, pyrogen-free physiological saline solution).

In some embodiments, the spinal cord injury is motor and/or sensory complete.

In some embodiments, the spinal cord injury is motor and/or sensory incomplete.

In some embodiments, the treating results in improved sensory function (e.g., reduced paresthesia, allodynia, and/or hyperalgesia).

In some embodiments, the treating results in improved motor function (e.g., locomotion, arm and hand function, coordination).

In some embodiments, the treating results in reduced bladder dysfunction (e.g., reduced neurogenic detrusor overactivity (NDO) and/or detrusor sphincter dyssynergia (DSD) through reduced external urethral sphincter activity (EUS EMG) during voiding).

In some embodiments, the subject is a human subject.

In some embodiments, the administering is carried out by injection.

In some embodiments, the administering is carried out by intravenous administration (e.g., intravenous injection). In some embodiments, the administering is carried out by intraperitoneal administration (e.g., intraperitoneal injection).

In some embodiments, the peptide is administered at a dosage of from 0.01 to 10 mg/kg (e.g., 0.05, 0.1, 0.5, 1, 2, 3, 4 or 5 mg/kg)).

In some embodiments, the peptide is administered in the acute phase of the spinal cord injury (e.g., within 30 minutes to 24 hours, or 2, 3, or 4 days following spinal cord injury).

In some embodiments, the peptide is administered in the subacute or chronic phase following the spinal cord injury (e.g., 2, 3, 4 or 5 weeks to one or more years following the spinal cord injury).

In some embodiments, the peptide is administered in combination with a rehabilitative (e.g., locomotor training) or engineering (e.g., spinal cord stimulation) intervention for the spinal cord injury.

### BRIEF DESCRIPTION OF THE DRAWINGS

FIG. 1 is a graph showing the LPS-induced TNFα release from mixed glia cells in vitro from treatments with the CN-105 peptide and a comparison pentapeptide, VR-53.

FIG. 2, panel A is a graph showing the concentration of CN-105 in uninjured, male CD-1 mice after a single intravenous injection bolus dose of 1.98 mg/kg. FIG. 2, panel B is a graph showing the CN-105 retention in the brain and the spinal cord, calculated by dividing the concentration at 24 hours (C₂₄) by the peak concentration (Cₘₐₓ), 24 hours after a single intravenous injection bolus dose of 1.98 mg/kg. FIG. 2, panel C is a graph showing the total CN-105 exposure in the brain and the spinal cord, calculated using the area under the curve (AUC), for 48 hours after a single intravenous injection bolus dose of 1.98 mg/kg.

FIG. 3 are graphs showing the locomotor performance recovery (via Basso Mouse Scale) in adult, female C57BL/6J mice after a moderate, incomplete SCI at the 9^{th} thoracic vertebrae. The mice were subsequently treated with either 2.0 mg/kg CN-105 or a control vehicle intravenously 30 minutes post-injury, and then intraperitoneally (IP) at 3 hours, 6 hours, and then every 24 hours post-injury for three days. FIG. 3, panel A is a graph showing the number of consistent, weight-supported hindlimb steps at 21-days post-injury; and FIG. 3, panel B is a graph showing the number of animals capable of performing coordinated locomotion at 21-days post-injury.

FIG. 4 is a series of microscopy images showing the spinal cord sections at the injury epicenter stained for glial fibrillary acidic protein (GFAP) in a mouse after a severe SCI.

FIG. 5, panel A is a graph showing the number of non-voiding bladder contractions during acute cystometry in urethane-anesthetized, female Sprague-Dawley rats that received spinal cord transection between the 9^{th} and 10^{th} thoracic vertebrae, followed by CN-105 (2 mg/kg; sci_cn105) or 0.9% saline (sci_saline) treatments at 30 minutes and 3 hours post injury by IV injection, and then every 24 hours by IP for 7 days. Control mice did not receive a spinal cord transection (intact).

FIG. 5, panel B is a graph showing the bladder capacity measured from a single-fill cystometric trial in urethane-anesthetized, female Sprague-Dawley rats that received spinal cord transection between the 9^{th} and 10^{th} thoracic vertebrae. Three baseline measurements were taken, and then three measurements were taken after administration of CN-105 (2 mg/kg; cn105).

### DETAILED DESCRIPTION

Provided herein is a method for improving functional outcomes of a spinal cord injury (e.g., motor, sensory, autonomic (e.g., bladder) function) in a subject in need thereof. Treatment as taught herein may be beneficial for anyone who is currently living with or who experiences a spinal cord injury, and may include individuals with a motor and/or sensory complete (no preserved function below the level of spinal cord damage) or incomplete (some preserved function below the level of damage) spinal cord injury. In some embodiments, the spinal cord injury is an acute injury.

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

The disclosures of all patent references cited herein are hereby incorporated by reference to the extent they are consistent with the disclosure set forth herein. In case of a conflict in terminology, the present specification is controlling.

As used herein in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±10%, 5%, 1%, 0.5%, or even ±0.1% of the specified amount.

A therapeutically “effective” amount as used herein is an amount that provides some improvement or benefit to the subject. Alternatively stated, a “therapeutically effective” amount is an amount that will provide some alleviation, mitigation, or decrease in at least one clinical symptom in the subject (e.g., in the case of a spinal cord and/or neuronal injury reducing neuroinflammation, improving motor function, improving sensory function, improving autonomic or bladder function, reducing oxidative stress, reducing glial activation, increase the release of pro-inflammatory cytokines, etc.). Those skilled in the art will appreciate that the therapeutic effects need not be complete or curative, as long as some benefit is provided to the subject.

“Pharmaceutically acceptable,” as used herein, means a material that is not biologically or otherwise undesirable, i.e., the material can be administered to an individual along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; 21^{st} ed. 2005). Exemplary pharmaceutically acceptable carriers for the compositions of this invention include, but are not limited to, phosphate buffered saline (PBS), sterile pyrogen-free water, and other sterile pyrogen-free physiological saline solutions.

The term “administering” or “administration” of a composition of the present invention to a subject includes any route of introducing or delivering to a subject a compound to perform its intended function (e.g., for improving functional outcomes of a SCI in a subject).

A “peptide” as used herein refers to a compound that comprises amino acid residues (and/or amino acid mimetic(s)), or a derivative thereof. Amino acids are well known in the art and include, for example, isoleucine, leucine, alanine, asparagine, glutamine, lysine, aspartic acid, glutamic acid, methionine, cysteine, phenylalanine, threonine, tryptophan, glycine, valine, proline, serine, tyrosine, arginine, histidine, norleucine, ornithine, taurine, selenocysteine, selenomethionine, lanthionine, 2-aminoisobutyric acid, dehydroalanine, hypusine, citrulline, 3-aminopropanoic acid, gamma-aminobutryic acid, nitroarginine, N-methylated leucine, homoarginine, dimethyl arginine, acetyl lysine, azalysine, pyrrolysine, and the like. An “amino acid side chain” refers to the various organic substituent groups that differentiate one amino acid from another. An amino acid having a hydrophobic side chain includes the non-limiting examples of alanine (A), isoleucine (I), leucine (L), methionine (M), phenylalanine (F), tryptophan (W), tyrosine (Y), and valine (V). An amino acid having a positively charged side chain, under typical physiological conditions, includes the non-limiting examples of arginine (R), histidine (H), and lysine (K). An amino acid having a negatively charged side chain, under typical physiological conditions, includes the non-limiting examples of aspartic acid (D) and glutamic acid (E). An amino acid having a polar uncharged side chain includes the non-limiting examples of serine (S), threonine (T), asparagine (N), and glutamine (Q). A “derivative” of an amino acid side chain refers to an amino acid side chain that has been modified structurally (e.g., through chemical reaction to form a new species, covalent linkage to another molecule, and the like). Some embodiments provide for a peptide comprising modifications including, but not limited to, glycosylation, side chain oxidation, acetylation, amidation, or phosphorylation, as long as the modification does not destroy the biological activity of the peptides as herein described. For example, in some embodiments, a peptide may be modified by N-terminal acetylation and/or C-terminal amidation.

An “amino acid mimetic” as used herein is meant to encompass peptidomimetics, peptoids (poly-N-substituted glycines) and β-peptides (i.e., peptides that comprise one or more amino acids residues having the amino group attached at the β-carbon rather than the α-carbon). Suitably, the amino acid mimetic comprises an altered chemical structure that is designed to adjust molecular properties favorably (e.g., stability, activity, reduced immunogenic response, solubility, etc.). Typically, the altered chemical structure is thought to not occur in nature (e.g., incorporating modified backbones, non-natural amino acids, etc.). Thus, non-limiting examples of amino acid mimetic include D-peptides, retro-peptides, retro-inverso peptides, β-peptides, peptoids, and compounds that include one or more D-amino acids, poly-N-substituted glycine, or β-amino acid, or any combination thereof.

Typically, a peptide comprises a sequence of at least 3 amino acids (amino acid residues) and/or amino acid mimetics. Embodiments of the disclosure relate to small peptides of at least 3, 4, 5, 6, 7, 8, or 9 amino acid residues, mimetics, or combinations thereof. Some embodiments described herein provide for peptides of fewer than 9, 8, 7, 6, 5, or 4 amino acid residues and/or mimetics. Some embodiments relate to peptides that are 5 amino acids in length. The peptides described herein can be provided in a charged form, typically with a net positive charge, and can be generated and used as salts (e.g., alkali metal salts, basic or acidic addition salts). The selection and formation of such salts are within the ability of one skilled in the art. See, e.g., Remington: The Science and Practice of Pharmacy, 21st ed., Lippincott Williams & Wilkins, A Wolters Kluwer Company, Philadelphia, Pa (2005).

Embodiments of the disclosure provide synthetic peptides with ApoE mimetic activity (e.g., have ApoE receptor-binding domain activity). Though they may exhibit ApoE mimetic activity, the disclosed peptides do not share primary protein sequence identity with the native ApoE polypeptide. In other words, the disclosed peptide sequences do not appear in the primary amino acid sequence of an ApoE polypeptide, nor do they exhibit α-helical secondary structure analogous to the native ApoE receptor-binding domain. In an embodiment, the synthetic peptides are optionally isolated and/or purified to a single active species.

In an aspect of the disclosure, the peptide comprises Formula I:

X1-X2-X3-X4-X5 (SEQ ID NO:1)

or a salt thereof, wherein X1 is selected from an amino acid having a hydrophobic side chain or an amino acid having a positively charged side chain; X2 is selected from an amino acid having a hydrophobic side chain, an amino acid having a positively charged side chain, or an amino acid having a polar uncharged side chain; X3 is selected from an amino acid having a positively charged side chain; X4 is selected from an amino acid having a positively charged side chain; and X5 is selected from an amino acid having a hydrophobic side chain or an amino acid having a positively charged side chain. Some embodiments provide a peptide wherein X1 is V or R; X2 is S, A, or H; X3 is K or R; X4 is K or R; and X5 is R, L, or K.

Some embodiments of this aspect provide for a peptide of Formula II:

X1-X2-X3-X4-X5-X6-X7-X8-X9 (SEQ ID NO:13);

wherein X1, X2, X3, X4, and X5 are as noted above, and each of X6, X7, X8, and X9 are independently selected from any amino acid, and are optionally absent. In such embodiments, the peptide of Formula II can include 5 amino acid residues, 6 amino acid residues, 7 amino acid residues, 8 amino acid residues, or 9 amino acid residues.

A number of non-limiting embodiments of peptides according to Formula I are disclosed in Table 1. In some embodiments the peptide can comprise VSRKR (SEQ ID NO:2), VSKRR (SEQ ID NO:3), VSRRR (SEQ ID NO:4), VARKL (SEQ ID NO:5), RHKKL (SEQ ID NO:6), RARRL (SEQ ID NO:7), RSKKL (SEQ ID NO:8), RHKRR (SEQ ID NO:9), VARRL (SEQ ID NO:10), VARRK (SEQ ID NO:11), or RSKRR (SEQ ID NO:12). In some embodiments, the peptide according to Formula I is VSRKR (SEQ ID NO:2), VSKRR (SEQ ID NO:3), VSRRR (SEQ ID NO:4), VARKL (SEQ ID NO:5), RHKKL (SEQ ID NO:6), RARRL (SEQ ID NO:7), RSKKL (SEQ ID NO:8), RHKRR (SEQ ID NO:9), VARRL (SEQ ID NO:10), VARRK (SEQ ID NO:11), or RSKRR (SEQ ID NO:12).

In some embodiments of all the aspects described herein, the peptides consist essentially of the amino acid sequences and formulae disclosed herein. In some embodiments of all the aspects described herein, the peptides consist of the amino acid sequences and formulae disclosed herein. See also U.S. Pat. Nos. 9,018,169 and 9,303,063 to Laskowitz et al.

TABLE 1
ApoE mimetic peptide sequences.

| Peptide | Sequence | | | | |
|---|---|---|---|---|---|
| SEQ ID NO: 1 | X1 | X2 | X3 | X4 | X5 |
| SEQ ID NO: 2 | V | S | R | K | R |
| SEQ ID NO: 3 | V | S | K | R | R |
| SEQ ID NO: 4 | V | S | R | R | R |
| SEQ ID NO: 5 | V | A | R | K | L |
| SEQ ID NO: 6 | R | H | K | K | L |
| SEQ ID NO: 7 | R | A | R | R | L |
| SEQ ID NO: 8 | R | S | K | K | L |
| SEQ ID NO: 9 | R | H | K | R | R |
| SEQ ID NO: 10 | V | A | R | R | L |
| SEQ ID NO: 11 | V | A | R | R | K |
| SEQ ID NO: 12 | R | S | K | R | R |

In some embodiments, the peptides can exhibit at least one ApoE mimetic activity. In some embodiments, for example, the disclosed peptides can bind one or more physiological ApoE receptors such as, for example, cell-surface receptors expressed by glial cells, as well as receptors that function to suppress the neuronal cell death and calcium influx (excitotoxicity) associated with N-methyl-D-aspartate (NMDA) exposure; protect against LPS-induced production of TNF-α and IL-6 (e.g., in an in vivo sepsis model); prevent, treat, or slow inflammatory disorders such as atherosclerosis, arthritis, or inflammatory bowel disease; suppress glial or microglial activation; suppress macrophage activation; suppress lymphocyte activation; suppress inflammation; suppress CNS inflammation; suppress lactate dehydrogenase release; treat neuropathy; and/or ameliorate neuronal injury in neurodegenerative disease (e.g., mild cognitive impairment, dementia, Parkinson's disease, or Alzheimer's disease) and/or acute CNS trauma (e.g., traumatic brain injury).

In some embodiments, a peptide as taught herein binds to and/or interacts with known ApoE interactions partners, such as SET protein (e.g., inhibitor 2 of protein phosphatase 2A (I2PP2A).

In some embodiments, the peptides are able to pass through the blood-brain barrier and/or the blood-spinal cord barrier. In some embodiments, the peptides are able to pass through the blood-brain barrier and/or the blood-spinal cord barrier without a chaperone.

The peptides can be produced using any means for making polypeptides known in the art, including, e.g., synthetic and recombinant methods. For example, in some embodiments the peptides can be synthesized using synthetic chemistry techniques such as solid-phase synthesis, Merrifield-type solid-phase synthesis, t-Boc solid-phase synthesis, Fmoc solid-phase synthesis, BOP solid-phase synthesis, and solution-phase synthesis. See, for example, Stewart and Young, Solid Phase Peptide Synthesis, 2nd ed., (1984) Pierce Chem. Co., Rockford Ill.; The Peptides: Analysis, Synthesis, Biology, Gross and Meienhofer, Eds., vols. 1-2 (1980) Academic Press, New York; Bodansky, Principles of Peptide Synthesis, (1984) Springer-Verlag, Berlin. In other embodiments, the peptides can be produced, for example, by expressing the peptide from a nucleic acid encoding the peptide in a cell or in a cell-free system according to recombinant techniques familiar to those of skill in the art. See, e.g., Sambrook et al., Molecular Cloning: A Laboratory Manual, (2001) Cold Spring Harbor Laboratory Press, Cold Spring Harbor, NY; Ausubel et al., Current Protocols in Molecular Biology, (2002) John Wiley & Sons, Somerset, NJ; each of which is hereby incorporated by reference in its entirety. The peptides can incorporate any of the various modifications and protective groups described herein or otherwise known to those of skill in the art, such as, for example, those described in McOmie, Protective Groups in Organic Chemistry, (1973) Plenum Press, New York.

Particular embodiments of peptide active agents as described above (shown in the form of their chemical structures), and for use in the methods and compositions described herein include, but are not limited to, the following, along with pharmaceutically acceptable salts thereof:

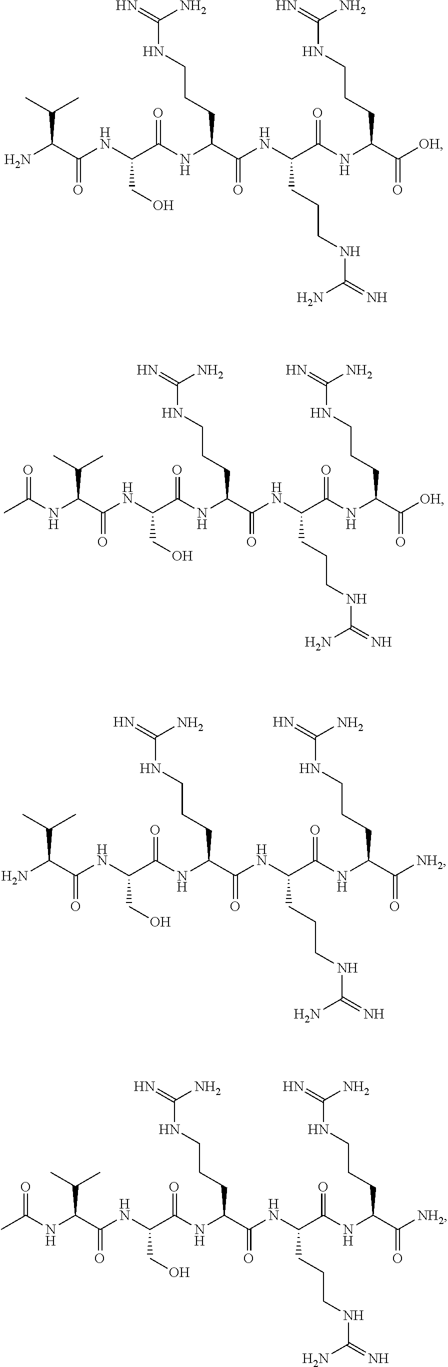 [SMILES: CC(C)[C@H](N)C(=O)N[C@@H](CO)C(=O)N[C@@H](CCCNC(=N)N)C(=O)N[C@@H](CCCNC(=N)N)C(=O)N[C@@H](CCCNC(=N)N)C(=O)O ; CC(=O)N[C@H](C(=O)N[C@@H](CO)C(=O)N[C@@H](CCCNC(=N)N)C(=O)N[C@@H](CCCNC(=N)N)C(=O)N[C@@H](CCCNC(=N)N)C(=O)O)C(C)C ; CC(C)[C@H](N)C(=O)N[C@@H](CO)C(=O)N[C@@H](CCCNC(=N)N)C(=O)N[C@@H](CCCNC(=N)N)C(=O)N[C@@H](CCCNC(=N)N)C(N)=O ; CC(=O)N[C@H](C(=O)N[C@@H](CO)C(=O)N[C@@H](CCCNC(=N)N)C(=O)N[C@@H](CCCNC(=N)N)C(=O)N[C@@H](CCCNC(=N)N)C(N)=O)C(C)C]

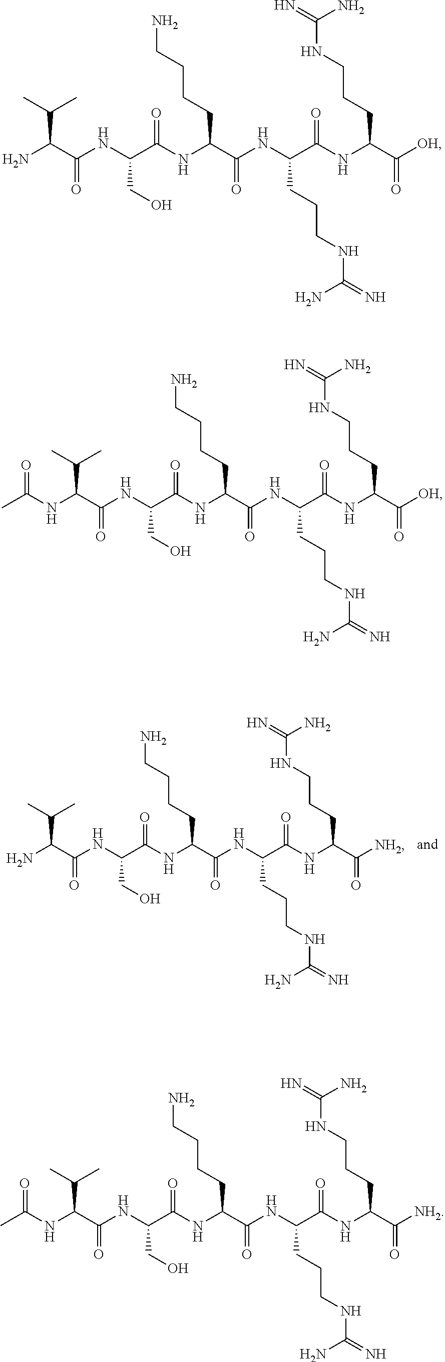 [SMILES: CC(C)[C@H](N)C(=O)N[C@@H](CO)C(=O)N[C@@H](CCCCN)C(=O)N[C@@H](CCCNC(=N)N)C(=O)N[C@@H](CCCNC(=N)N)C(=O)O ; CC(=O)N[C@H](C(=O)N[C@@H](CO)C(=O)N[C@@H](CCCCN)C(=O)N[C@@H](CCCNC(=N)N)C(=O)N[C@@H](CCCNC(=N)N)C(=O)O)C(C)C ; CC(C)[C@H](N)C(=O)N[C@@H](CO)C(=O)N[C@@H](CCCCN)C(=O)N[C@@H](CCCNC(=N)N)C(=O)N[C@@H](CCCNC(=N)N)C(N)=O ; CC(=O)N[C@H](C(=O)N[C@@H](CO)C(=O)N[C@@H](CCCCN)C(=O)N[C@@H](CCCNC(=N)N)C(=O)N[C@@H](CCCNC(=N)N)C(N)=O)C(C)C]

Subjects described herein include mammalian subjects, including both human subjects and non-human (animal) subjects, though in some embodiments human subjects are preferred. Subjects may also include non-human primates, rodents such as mice and rats, dogs, cats, rabbits, goats, horses, pigs, cattle, etc., for research and/or veterinary purposes.

Subjects include both male and female subjects and subjects of all ages including infant, juvenile, adolescent and adult subjects. In some embodiments, subjects are infant or juvenile subjects.

A “subject in need” of the methods of the invention can be any subject known to have, or suspected of having increased risk of developing, an injury or disorder as described herein to which administering a peptide as described herein may provide beneficial health effects.

In some embodiments, the disclosed peptides and compositions may be administered by any suitable route of administration, including, but not limited to, injection (subcutaneous, intraperitoneal, intravenous, intrathecal, intramuscular, intracerebroventricular, and spinal injection), intranasal, oral, transdermal, parenteral, inhalation, nasopharyngeal or transmucosal absorption. In some embodiments, the peptides may be delivered by injection, inhalation, transdermal, intravenous, intranasal, intracranial, and/or intrathecal administration. In some embodiments, the peptides may be delivered intravenously (e.g., provided in normal saline). Administration encompasses the providing at least one peptide as described herein (e.g., of Formula I, Formula II, and/or SEQ ID NOs:1-12) formulated as a pharmaceutical composition. Administration of an active agent (e.g., compound, peptide, etc.) directly to the brain is known in the art. Intrathecal injection delivers agents directly to the brain ventricles and the spinal fluid. Surgically-implantable infusion pumps are available to provide sustained administration of agents directly into the spinal fluid. Spinal injection involves lumbar puncture with injection of a pharmaceutical compound into the cerebrospinal fluid. Administration also includes targeted delivery wherein peptide according to the disclosure is active only in a targeted region of the body (for example, in brain and/or spinal cord tissue), as well as sustained release formulations in which the peptide is released over a period of time in a controlled manner. Sustained release formulations and methods for targeted delivery are known in the art and include, for example, use of liposomes, drug loaded biodegradable microspheres, drug-polymer conjugates, drug-specific binding agent conjugates and the like.

Pharmaceutically acceptable carriers are well known to those of skill in the art and may include, but are not limited to, sterile pyrogen-free water, sterile pyrogen-free physiological saline solution, etc. Pharmaceutically acceptable carriers may also include chitosan nanoparticles or other related enteric polymer formulations.

Particular pharmaceutical formulations and therapeutically effective amounts and dosing regimens may be determined by one of skill in the art based upon the disclosures herein and taking into consideration, for example, a subject's age, weight, sex, ethnicity, organ (e.g., liver and kidney) function, the extent of desired treatment, the stage and severity or risk of the disease or disorder and associated symptoms, and the tolerance of the subject. In some embodiments, effective amounts of the peptides disclosed herein can be about 0.1 or 0.5 to 5, 8 or 10 mg/kg (e.g., about 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9 or about 10 mg/kg), for example, about 1 mg/kg. In some embodiments, dosage regimens may include, but are not limited to, dosages once every 1, 2, 3, 4, 5, 6, 7 or 8 hours, for about 12, 24, 36, 48, 60, 72, or about 90 (e.g., about 12, 13, 14, 15, 16, 17, 18, 19, 20, 21, 22, 23, 24, 28, 32, 36, 40, 44, 48, 54, 60, 66, 72, 78, 84, or about 90 hours) hours after insult or injury. In some embodiments, dosage regimens may last for about 1 to about 14 days (e.g., about 1, 2, 3, 4, 5, 6, 7, 8, 9, 10, 11, 12, 13, or about 14 days). For example, the peptides may be administered once every 6 hours for 72 hours (13 doses total). In some embodiments, the peptides may be administered once every 24 hours for about 7 days or about 14 days.

In some embodiments, administering the peptides may comprise initially administering the peptides (e.g., one or more loading dose; e.g., administering during the acute phase of an injury, such as a SCI) followed by subsequent administration of the peptides (e.g., one or more maintenance doses; e.g., administering during the maintenance, subacute, or chronic phase of an injury, such as an SCI). In some embodiments, the peptides may be administered during the acute phase of an injury within about 15 minutes to about 180 minutes (e.g., about 15, 20, 25, 30, 35, 40, 45, 50, 55, 60, 75, 90, 105, 120, 135, 150, 165, or about 180 minutes) of the injury. In some embodiments, the peptides may be initially administered within about 30 minutes of the injury. In some embodiments, the peptides may be initially administered within about 30 minutes of the injury and within about 1 hour to about 6 hours (e.g., about 1, 2, 3, 4, 5, or about 6 hours) of the injury (e.g., the initial administration comprises two administrations, e.g., one or more loading dose). In some embodiments, the subsequent administration comprises administration of the peptides during the maintenance phase of the injury once every 24 hours from about 3 days to about 14 days (e.g., about 3, 4, 5, 6, 7, 8, 9, 10, 11, 12, 13, or about 14 days). In some embodiments, the subsequent administration comprises administration of the peptides during the maintenance phase of the injury about once every 1 day to about once every 7 days (e.g., about once every 1, 2, 3, 4, 5, 6, or about 7 days) for about 2 weeks to about 52 weeks (e.g., about 2, 3, 4, 5, 6, 7, 8, 9, 10, 12, 14, 16, 18, 20, 24, 28, 32, 36, 40, 44, 48, or about 52 weeks). In some embodiments, the initial administration (e.g., the one or more loading doses) is an intravenous administration. In some embodiments, the subsequent administration (e.g., the maintenance dose) is an intraperitoneal administration.

In some embodiments, the peptides may be administered prior to (e.g., in anticipation of) an injury (e.g., CNS trauma; e.g., a SCI). In some embodiments, the peptides may be administered from about 6 hours to about 1 hour (e.g., about 6, 5, 4, 3, 2, or about 1 hour) prior to an injury. In some embodiments, the peptides may be administered from about 60 minutes to about 10 minutes (e.g., about 60, 50, 40, 30, 20, or about 10 minutes) prior to an injury.

In some embodiments, the peptides may be administered in combination with a physical intervention for an injury (e.g., CNS trauma; e.g., an SCI). In some embodiments, the physical intervention is a rehabilitative (e.g., locomotor training and/or physical therapy) or an engineering (e.g., spinal cord stimulation) intervention.

In aspects described herein that relate to compositions, including pharmaceutical compositions and formulations, some embodiments provide a composition that comprises at least one peptide according to SEQ ID NO:1 (e.g., SEQ ID NOs:1-12), or a salt thereof (e.g., an acetate salt) in combination with an acceptable carrier.

Provided herein are methods of treatment for improving functional outcomes of a spinal cord injury (SCI) in a subject in need thereof, with administration of a peptide as taught herein. Such functional outcomes may include, but are not limited to, motor, sensory, and autonomic physiological functions. For example, the treatment as taught herein may result in improved motor function (e.g., locomotion, arm and hand function, coordination, etc.), improved sensory function (e.g., reduced paresthesia, allodynia, and/or hyperalgesia, etc.), and/or reduced autonomic dysfunction, such as reduced bladder dysfunction (e.g., reduced neurogenic detrusor overactivity (NDO) and/or detrusor sphincter dyssynergia (DSD) through reduced external urethral sphincter activity (EUS EMG) during voiding).

Treatment as taught herein may be beneficial for a subject who is suffering from a spinal cord injury, and may include individuals with a motor and/or sensory complete (no preserved function below the level of spinal cord damage) or incomplete (some preserved function below the level of damage) spinal cord injury.

The spinal cord injury may be an acute injury (e.g., within 30 minutes to 24 hours, or 2, 3, or 4 days following spinal cord injury), or in the subacute or chronic phase following spinal cord injury (e.g., 2, 3, 4 or 5 weeks to one or more years following spinal cord injury).

The present invention is explained in greater detail in the following non-limiting examples.

### Examples

### ApoE Mimetic Peptide Treatment of Motor, Sensory, Bladder and Other Associated Dysfunctions Caused by Spinal Cord Injury

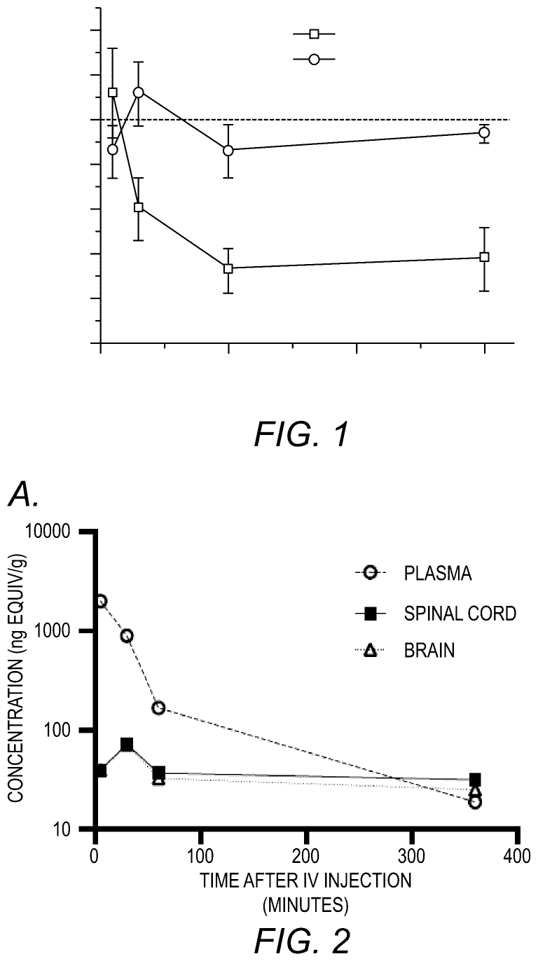

Despite the neuroprotective effectiveness of early generation apoE-mimetic peptides based on the primary structure of the apoE receptor-binding region, several factors have limited clinical translation. Among those factors are peptide size and production cost. In order to address these barriers, we developed a library of smaller, 5-amino acid peptides by linearizing the polar face of the receptor-binding region, which preserves peptide function without maintaining any primary sequence homology with the apoE receptor-binding region itself. Overall, 13 peptides were screened using in vitro assays of neuroinflammation and neuroprotection from excitotoxicity. The lead candidate peptide, CN-105, was selected based on its ability to suppress LPS-induced inflammation (i.e., TNFα) from a mixed glial culture and confer neuroprotection, defined as a reduction in neuronal lactate dehydrogenase (LDH) from a mixed neuronal culture (Table 2). The resulting smaller, 5-amino acid peptide increases the translational and therapeutic potential by reducing production cost, improving pharmacokinetics, and increasing CNS bioavailability. A comparison of the neuroprotective activity of CN-105 with VR-53, another pentapeptide from this library, as measured by LPS-induced inflammation from TNFα is shown in FIG. 1.

TABLE 2
Reduction in neuronal LDH release from a mixed
neuronal culture following incubation with NMDA
upon administration of peptides in vitro.

| | | | SEQ |
|---|---|---|---|
| Peptide at 1 μM | | Neuroprotection | ID NO |
| CN-105 | Ac-VSRRR-NH2 | 19.14% | 4 |
| (VR-55) | | | |
| VR-54 | Ac-VSKKR-NH2 | −15.91% | 14 |
| VR-53 | Ac-VSKRR-NH2 | 7.03% | 3 |
| VR-52 | Ac-VSRKR-NH2 | 12.63% | 2 |
| RL-S-3 | Ac-RSKKL-NH2 | −20.80% | 8 |
| RL-S-2 | Ac-RARRL-NH2 | −5.23% | 7 |
| RL-S-1 | Ac-RHKKL-NH2 | −2.85% | 6 |
| RR-S-2 | Ac-RSKRR-NH2 | 0.08% | 12 |
| RR-S-1 | Ac-RHKRR-NH2 | 7.5% | 9 |
| VL-S-3 | Ac-VARRL-NH2 | 11.50% | 10 |
| VL-S-1 | Ac-VARKL-NH2 | 24.48% | 5 |
| AL-10-1 | Ac-ASHLRKLRKRLL-NH2 | −6.21% | 15 |

We have since demonstrated that brain exposure to CN-105 is up to five times higher than previous peptides and improves both histological and behavioral outcomes following acquired brain injury (Laskowitz et al., 2017; Lei et al., 2016; Liu et al., 2018). Moreover, preclinical efficacy and a successful Phase I safety, tolerability, and pharmacokinetics study (Guptill et al., 2017) has led to CN-105 being approved for Orphan Drug (Designation Number: 14-4490) and Investigational New Drug (IND) status by the United States Food and Drug Administration.

### Pharmacokinetics and Spinal Cord Access of CN-105

While we have repeatedly shown the ability of apoE-mimetic peptides, including CN-105, to cross the blood-brain barrier, the ability and pharmacokinetics of spinal cord access is largely unknown. This is important, because while the BSCB serves the same general purpose as the blood-brain barrier, there are compelling data to suggest it is quite different (Bartanusz et al., 2011). For instance, when compared to the blood-brain barrier, the BSCB exhibits a decrease in the number of and vascular area coverage by pericytes (Winkler et al., 2012). In addition, the BSCB also has decreased amounts of transporter molecules (permeability-glycoprotein), tight junction proteins (zonula occludens-1, occludin), and adherens junction proteins (vascular endothelial cadherin, b-catenin) ultimately rendering it more permeable than the blood-brain barrier (Bartanusz et al., 2011; Winkler et al., 2012). These observed structural alterations ultimately result in greater basal BSCB permeability to passive tracers (Prockop et al., 1995; Winkler et al., 2012) and certain cytokines, including interferon-a, interferon-gamma (g), and TNFα (Pan & Kastin, 2008). Structural differences between brain and spinal cord barriers also correspond to increased peripheral immune cell infiltration into the spinal cord than brain following trauma (Schnell et al., 1999). These differences between the blood-brain and BSCB suggest that there may be differences in the either the ability or timing of CN-105 access to the spinal cord parenchyma.

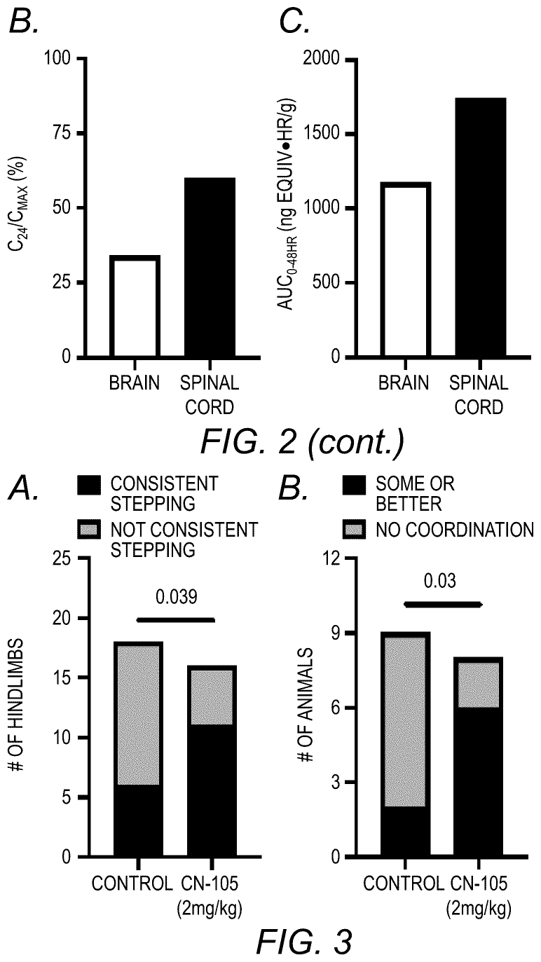

In collaboration with XenoBiotic Laboratories, Inc., we conducted a pharmacokinetic evaluation of radiolabled CN-105 in uninjured, male CD-1 mice. After a single intravenous bolus dose of CN-105 (1.98 mg/kg), peak concentration of CN-105 in the spinal cord occurred 30 minutes post injection (FIG. 2, panel A). The timing of CN-105 entry into the nervous system and maximal concentration was similar between brain and spinal cord regions (FIG. 2, panel A).

However, tissue retention of CN-105 at 24 hours (FIG. 2, panel B) and total exposure (FIG. 2, panel C) to CN-105 over 48 hours post injection was greatest in the spinal cord. Tissue retention at 24 hours post injection was quantified by dividing the CN-105 concentration at 24 hours (C₂₄) by the peak CN-105 concentration (Cₘₐₓ) and multiplying by 100. Using this metric, we saw that the spinal cord retained a higher percentage of CN-105 than the brain at 24 hours post injection (FIG. 2, panel B).

The total exposure to CN-105 was calculated using the area under the curve (AUC) from the time of injection to 48 hours post injection (AUC₀₋₄₈) and is expressed in nanogram equivalents*hour per gram. Over the course of 48 hours following intravenous injection, the spinal cord had greater total exposure to CN-105 than the brain (FIG. 2, panel C). Increased CN-105 access to the spinal cord is consistent with greater passive permeability in the intact spinal cord (Winkler et al., 2012). These data also suggest that CN-105 may have equal or more potential to confer benefit after SCI than in brain injury.

Preclinical Efficacy of CN-105 to Treat Motor and Sensory Dysfunction after SCI

To determine whether CN-105 can improve sensorimotor outcomes following a moderate, incomplete SCI, we conducted experiments in adult, female C57BL/6J mice. Here, we delivered CN-105 (2.0 mg/kg) or Vehicle at 30 minutes (intravenous, IV), at 3 hours and 6 hours (intraperitoneal, IP), then every 24 hours (IP) for three days following a moderate, contusive SCI at the level of the 9th thoracic vertebrae (T9). We tracked locomotor recovery using the Basso Mouse Scale (BMS) (Basso et al., 2006) and assessed for neuropathic pain or allodynia using the von Frey hair test. Treatment with 2.0 mg/kg CN-105 after moderate SCI resulted in significantly more animals capable of consistently taking weight-supported steps (p<0.05) and performing coordinated locomotion (p<0.05) at 21 days after injury (FIG. 3). There was also a trend toward improved sensory function, or reduction in allodynia, in mice treated with CN-105.

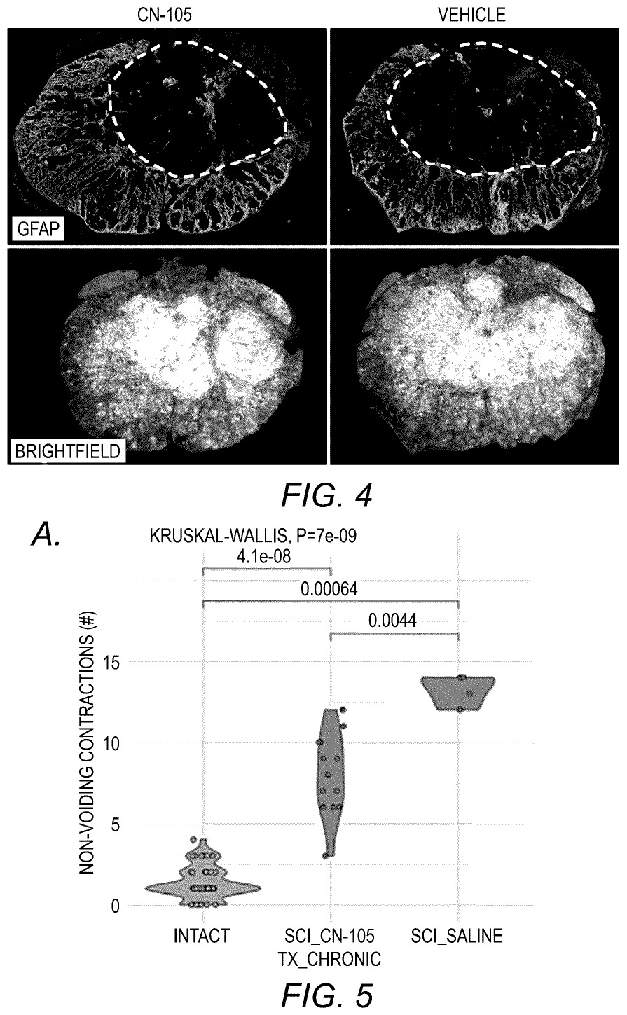

In a separate experiment, we delivered CN-105 (0.05 mg/kg) before and after SCI. To determine the effect of CN-105 treatment on neuropathology 14 days after severe SCI, we stained spinal cord sections at the injury epicenter for glial fibrillary acidic protein (GFAP; FIG. 4), which labels astrocytes, including those that form the glial scar around the injury. In this approach, spared tissue can be differentiated from the lesion core by the presence (spared tissue) or absence (lesion core) of GFAP staining (Duncan et al., 2018). Treatment with CN-105 before and after injury appeared to reduce the lesion size and tissue damage caused by a severe, contusive SCI, thus increasing tissue sparing (FIG. 4).

Preclinical Efficacy of CN-105 to Treat Bladder Dysfunction after SCI

Without wishing to be bound by any particular theory, given that CN-105 binds α7 nicotinic acetylcholine receptors (Xue et al., 2022), which are heavily expressed in the urothelium, it is feasible that CN-105 could improve post-SCI bladder dysfunction. Thus, we administered a treatment of CN-105 in order to reduce neurogenic detrusor overactivity (NDO) and suppress detrusor sphincter dyssynergia (DSD) through reduced external urethral sphincter activity (EUS EMG) during voiding, ultimately increasing the efficiency of bladder emptying.

We conducted terminal acute experiments in urethane-anesthetized, female Sprague-Dawley rats to quantify the chronic and acute effects of CN-105 on urodynamic function. Rats (n=6) received spinal cord transection (TX) at T9/T10 followed by CN-105 (2.0 mg/kg) or 0.9% saline treatment at 30 min and 3 hours (IV), then every 24 hours (IP) for 7 days post-injury. Collection of body weight and void volumes occurred 2-3 times daily during manual bladder expression throughout the duration of the study. For acute cystometry experiments, we placed a catheter into the bladder dome to measure pressure and an electromyography paddle on the EUS to measure muscle activity. During a single-fill cystometric trial, we infused the bladder with saline at a constant rate (54-84 ml/hr) until a distension-evoked bladder contraction voided urine or until overflow incontinence occurred. Following 3 baseline cystometric trials, we performed 3 trials after administration of CN-105.

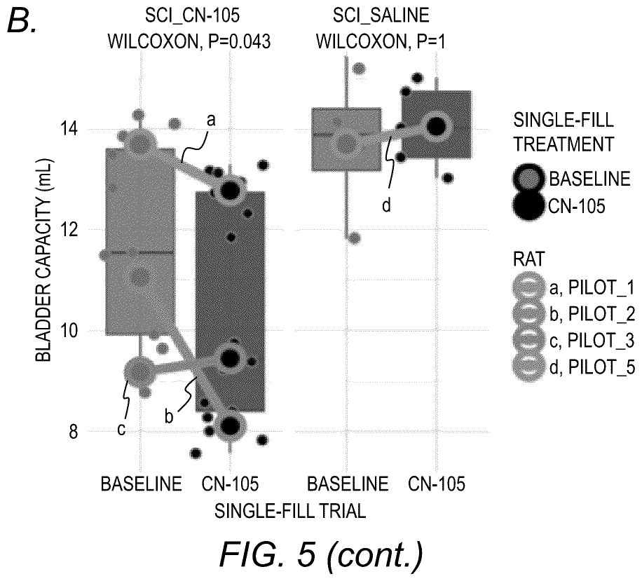

Treatment with CN-105 prevented post-injury weight loss consistent with previous generation ApoE mimetic peptides. In agreement with the literature, SCI rats demonstrated increased bladder capacities (BC; CN105: 11.83±2.00; Saline: 13.69±1.42 mL), decreased voiding efficiency (VE; CN105: 3.31±1.83; Saline: 5.76±1.87%), frequent non-voiding contractions (NVCs: CN105: 8.0±2.48; Saline: 13.25±0.96) vs. intact (BC: 0.82±0.43 mL; VE: 23.34±17.61%; NVC: 1.35±1.02; Kruskal-Wallis p<0.05). Post-hoc analysis indicates that extended CN-105 treatment may decrease NDO (FIG. 5, panel A; p<0.05), and acute dosing of CN-105 may decrease bladder capacities in chronically treated rats (FIG. 5, panel B; p<0.05).

Taken together, these data demonstrated that CN-105 reduces neuropathology and improves functional outcomes in a clinically relevant small animal model of SCI. Without wishing to be bound by any particular theory, these data along with demonstrated preclinical efficacy of previous apoE-mimetic peptides provides confidence that CN-105, delivered in clinically meaningful windows before or after SCI, can improve post-injury outcomes and would be a tremendous asset to the SCI community.

### REFERENCES

1. Aono, M., Bennett, E. R., Kim, K. S., Lynch, J. R., Myers, J., Pearlstein, R. D., Warner, D. S., & Laskowitz, D. T. (2003). Protective effect of apolipoprotein E-mimetic peptides on N-methyl-D-aspartate excitotoxicity in primary rat neuronal-glial cell cultures. Neuroscience, 116(2), 437-445.
2. Armour, B. S., Courtney-Long, E. A., Fox, M. H., Fredine, H., & Cahill, A. (2016). Prevalence and Causes of Paralysis-United States, 2013. Am J Public Health, 106(10), 1855-1857.
3. Bartanusz, V., Jezova, D., Alajajian, B., & Digicaylioglu, M. (2011). The blood-spinal cord barrier: morphology and clinical implications. Ann Neurol, 70(2), 194-206.
4. Basso, D. M., Beattie, M. S., & Bresnahan, J. C. (1996). Graded histological and locomotor outcomes after spinal cord contusion using the NYU weight-drop device versus transection. Exp Neurol, 139, 244-256.
5. Basso, D. M., Fisher, L. C., Anderson, A. J., Jakeman, L. B., McTigue, D. M., & Popovich, P. G. (2006). Basso Mouse Scale for locomotion detects differences in recovery after spinal cord injury in five common mouse strains. J Neurotrauma, 23(5), 635-659.
6. Batchelor, P. E., Tan, S., Wills, T. E., Porritt, M. J., & Howells, D. W. (2008). Comparison of Inflammation in the Brain and Spinal Cord following Mechanical Injury. Journal of Neurotrauma, 25(10), 1217-1225.
7. Bellver-Landete, V., Bretheau, F., Mailhot, B., Vallieres, N., Lessard, M., Janelle, M. E., Vernoux, N., Tremblay, M. E., Fuehrmann, T., Shoichet, M. S., & Lacroix, S. (2019). Microglia are an essential component of the neuroprotective scar that forms after spinal cord injury. Nat Commun, 10(1), 518.
8. Boyles, J. K., Pitas, R. E., Wilson, E., Mahley, R. W., & Taylor, J. M. (1985). Apolipoprotein E associated with astrocytic glia of the central nervous system and with nonmyelinating glia of the peripheral nervous system. The Journal of Clinical Investigation, 76(4), 1501-1513.
9. Brennan, F. H., Hall, J. C. E., Guan, Z., & Popovich, P. G. (2018). Microglia limit lesion expansion and promote functional recovery after spinal cord injury in mice. bioRxiv, 410258.
10. Carlson, S. L., Parrish, M. E., Springer, J. E., Doty, K., & Dossett, L. (1998). Acute Inflammatory Response in Spinal Cord Following Impact Injury. Experimental Neurology, 151(1), 77-88.
11. Cheng, X., Zheng, Y., Bu, P., Qi, X., Fan, C., Li, F., Kim, D. H., & Cao, Q. (2018). Apolipoprotein E as a novel therapeutic neuroprotection target after traumatic spinal cord injury. Exp Neurol, 299(Pt A), 97-108.
12. DeJong, G., Tian, W., Hsieh, C. H., Junn, C., Karam, C., Ballard, P. H., Smout, R. J., Horn, S. D., Zanca, J. M., Heinemann, A. W., Hammond, F. M., & Backus, D. (2013). Rehospitalization in the first year of traumatic spinal cord injury after discharge from medical rehabilitation. Arch Phys Med Rehabil, 94 (4 Suppl 2), S87-97.
13. Donnelly, D. J., & Popovich, P. G. (2008). Inflammation and its role in neuroprotection, axonal regeneration and functional recovery after spinal cord injury. Experimental Neurology, 209(2), 378-388.
14. Duncan, G. J., Manesh, S. B., Hilton, B. J., Assinck, P., Liu, J., Moulson, A., Plemel, J. R., & Tetzlaff, W. (2018). Locomotor recovery following contusive spinal cord injury does not require oligodendrocyte remyelination. Nat Commun, 9(1), 3066.
15. Evans, T. A., Barkauskas, D. S., Myers, J. T., Hare, E. G., You, J. Q., Ransohoff, R. M., Huang, A. Y., & Silver, J. (2014). High-resolution intravital imaging reveals that blood-derived macrophages but not resident microglia facilitate secondary axonal dieback in traumatic spinal cord injury. Experimental Neurology, 254, 109-120.
16. Fleming, J. C., Norenberg, M. D., Ramsay, D. A., Dekaban, G. A., Marcillo, A. E., Saenz, A. D., Pasquale-Styles, M., Dietrich, W. D., & Weaver, L. C. (2006). The cellular inflammatory response in human spinal cords after injury. Brain: A Journal Of Neurology, 129(Pt 12), 3249-3269.
17. Gensel, J. C., Nakamura, S., Guan, Z., van Rooijen, N., Ankeny, D. P., & Popovich, P. G. (2009). Macrophages promote axon regeneration with concurrent neurotoxicity. J Neurosci, 29(12), 3956-3968.
18. Goulet, J., Richard-Denis, A., Thompson, C., & Mac-Thiong, J. M. (2019). Relationships Between Specific Functional Abilities and Health-Related Quality of Life in Chronic Traumatic Spinal Cord Injury. Am J Phys Med Rehabil, 98(1), 14-19.
19. Greenhalgh, A. D., & David, S. (2014). Differences in the phagocytic response of microglia and peripheral macrophages after spinal cord injury and its effects on cell death. The Journal Of Neuroscience: The Official Journal Of The Society For Neuroscience, 34(18), 6316-6322.
20. Greenhalgh, A. D., Zarruk, J. G., Healy, L. M., Baskar Jesudasan, S. J., Jhelum, P., Salmon, C. K., Formanek, A., Russo, M. V., Antel, J. P., McGavern, D. B., McColl, B. W., & David, S. (2018). Peripherally derived macrophages modulate microglial function to reduce inflammation after CNS injury. PLoS Biol, 16(10), e2005264.
21. Gu, Z., Li, F., Zhang, Y. P., Shields, L. B., Hu, X., Zheng, Y., Yu, P., Zhang, Y., Cai, J., Vitek, M. P., & Shields, C. B. (2013). Apolipoprotein E Mimetic Promotes Functional and Histological Recovery in Lysolecithin-Induced Spinal Cord Demyelination in Mice. J Neurol Neurophysiol, 2014(Suppl 12), 10.
22. Guptill, J. T., Raja, S. M., Boakye-Agyeman, F., Noveck, R., Ramey, S., Tu, T. M., & Laskowitz, D. T. (2017). Phase 1 Randomized, Double-Blind, Placebo-Controlled Study to Determine the Safety, Tolerability, and Pharmacokinetics of a Single Escalating Dose and Repeated Doses of CN-105 in Healthy Adult Subjects. J Clin Pharmacol, 57(6), 770-776.
23. Hansen, C. N., Fisher, L. C., Deibert, R. J., Jakeman, L. B., Zhang, H., Noble-Haeusslein, L., White, S., & Basso, D. M. (2013). Elevated MMP-9 in the lumbar cord early after thoracic spinal cord injury impedes motor relearning in mice. Journal of Neuroscience, 33(32), 13101-13111.
24. Hansen, C. N., Norden, D. M., Faw, T. D., Deibert, R., Wohleb, E. S., Sheridan, J. F., Godbout, J. P., & Basso, D. M. (2016). Lumbar Myeloid Cell Trafficking into Locomotor Networks after Thoracic Spinal Cord Injury. Experimental Neurology, 282, 86-98.
25. James, M. L., Blessing, R., Bennett, E., & Laskowitz, D. T. (2009). Apolipoprotein E modifies neurological outcome by affecting cerebral edema but not hematoma size after intracerebral hemorrhage in humans. J Stroke Cerebrovasc Dis, 18(2), 144-149.
26. James, S. L., Theadom, A., Ellenbogen, R. G., Bannick, M. S., Montjoy-Venning, W., Lucchesi, L. R., Abbasi, N., Abdulkader, R., Abraha, H. N., Adsuar, J. C., Afarideh, M., Agrawal, S., Ahmadi, A., Ahmed, M. B., Aichour, A. N., Aichour, I., Aichour, M. T. E., Akinyemi, R. O., Akseer, N., . . . Murray, C. J. L. (2019). Global, regional, and national burden of traumatic brain injury and spinal cord injury, 1990-2016: a systematic analysis for the Global Burden of Disease Study 2016. The Lancet Neurology, 18(1), 56-87.
27. Jha, A., Lammertse, D. P., Coll, J. R., Charlifue, S., Coughlin, C. T., Whiteneck, G. G., & Worley, G. (2008). Apolipoprotein E epsilon4 allele and outcomes of traumatic spinal cord injury. The journal of spinal cord medicine, 31(2), 171-176.
28. Kachourbos, M. J., & Creasey, G. H. (2000). Health promotion in motion: improving quality of life for persons with neurogenic bladder and bowel using assistive technology. SCI Nurs, 17(3), 125-129.
29. Kigerl, K. A., Gensel, J. C., Ankeny, D. P., Alexander, J. K., Donnelly, D. J., & Popovich, P. G. (2009). Identification of two distinct macrophage subsets with divergent effects causing either neurotoxicity or regeneration in the injured mouse spinal cord. The Journal Of Neuroscience: The Official Journal Of The Society For Neuroscience, 29(43), 13435-13444.
30. Lanterna, L. A., Ruigrok, Y., Alexander, S., Tang, J., Biroli, F., Dunn, L. T., & Poon, W. S. (2007). Meta-analysis of APOE genotype and subarachnoid hemorrhage: clinical outcome and delayed ischemia. Neurology, 69(8), 766-775.
31. Laskowitz, D. T., Goel, S., Bennett, E. R., & Matthew, W. D. (1997). Apolipoprotein E suppresses glial cell secretion of TNF alpha. J Neuroimmunol, 76(1-2), 70-74.
32. Laskowitz, D. T., Horsburgh, K., & Roses, A. D. (1998). Apolipoprotein E and the CNS response to injury. J Cereb Blood Flow Metab, 18(5), 465-471.
33. Laskowitz, D. T., Lei, B., Dawson, H. N., Wang, H., Bellows, S. T., Christensen, D. J., Vitek, M. P., & James, M. L. (2012). The apoE-mimetic peptide, COG1410, improves functional recovery in a murine model of intracerebral hemorrhage. Neurocrit Care, 16(2), 316-326.
34. Laskowitz, D. T., McKenna, S. E., Song, P., Wang, H., Durham, L., Yeung, N., Christensen, D., & Vitek, M. P. (2007). COG1410, a novel apolipoprotein E-based peptide, improves functional recovery in a murine model of traumatic brain injury. J Neurotrauma, 24(7), 1093-1107.
35. Laskowitz, D. T., Sheng, H., Bart, R. D., Joyner, K. A., Roses, A. D., & Warner, D. S. (1997). Apolipoprotein E deficient mice have increased susceptibility to focal cerebral ischemia. J Cereb Blood Flow Metab, 17(7), 753-758.
36. Laskowitz, D. T., Song, P., Wang, H., Mace, B., Sullivan, P. M., Vitek, M. P., & Dawson, H. N. (2010). Traumatic brain injury exacerbates neurodegenerative pathology: improvement with an apolipoprotein E-based therapeutic. J Neurotrauma, 27(11), 1983-1995.
37. Laskowitz, D. T., Thekdi, A. D., Thekdi, S. D., Han, S. K., Myers, J. K., Pizzo, S. V., & Bennett, E. R. (2001). Downregulation of microglial activation by apolipoprotein E and apoE-mimetic peptides. Exp Neurol, 167(1), 74-85.
38. Laskowitz, D. T., Wang, H., Chen, T., Lubkin, D. T., Cantillana, V., Tu, T. M., Kernagis, D., Zhou, G., Macy, G., Kolls, B. J., & Dawson, H. N. (2017). Neuroprotective pentapeptide CN-105 is associated with reduced sterile inflammation and improved functional outcomes in a traumatic brain injury murine model. Sci Rep, 74-85.
39. Lawrence, D. W., Comper, P., Hutchison, M. G., & Sharma, B. (2015). The role of apolipoprotein E episilon (F)-4 allele on outcome following traumatic brain injury: A systematic review. Brain Inj, 29(9), 1018-1031.
40. Lei, B., James, M. L., Liu, J., Zhou, G., Venkatraman, T. N., Lascola, C. D., Acheson, S. K., Dubois, L. G., Laskowitz, D. T., & Wang, H. (2016). Neuroprotective pentapeptide CN-105 improves functional and histological outcomes in a murine model of intracerebral hemorrhage. Sci Rep, 6, 34834.
41. Linton, M. F., Gish, R., Hubl, S. T., Butler, E., Esquivel, C., Bry, W. I., Boyles, J. K., Wardell, M. R., & Young, S. G. (1991). Phenotypes of apolipoprotein B and apolipoprotein E after liver transplantation. J Clin Invest, 88(1), 270-281.
42. Liu, J., Zhou, G., Kolls, B. J., Tan, Y., Fang, C., Wang, H., & Laskowitz, D. T. (2018). Apolipoprotein E mimetic peptide CN-105 improves outcome in a murine model of SAH. Stroke Vasc Neurol, 3(4), 222-230.
43. Lynch, J. R., Tang, W., Wang, H., Vitek, M. P., Bennett, E. R., Sullivan, P. M., Warner, D. S., & Laskowitz, D. T. (2003). APOE genotype and an ApoE-mimetic peptide modify the systemic and central nervous system inflammatory response. J Biol Chem, 278(49), 48529-48533.
44. Ma, M., Basso, D. M., Walters, P., Stokes, B. T., & Jakeman, L. B. (2001). Behavioral and histological outcomes following graded spinal cord contusion injury in the C57Bl/6 mouse. Exp Neurol, 169(2), 239-254.
45. Mahley, R. W. (1988). Apolipoprotein E: cholesterol transport protein with expanding role in cell biology. Science, 240(4852), 622-630.
46. Misra, U. K., Adlakha, C. L., Gawdi, G., McMillian, M. K., Pizzo, S. V., & Laskowitz, D.

T. (2001). Apolipoprotein E and mimetic peptide initiate a calcium-dependent signaling response in macrophages. J Leukoc Biol, 70(4), 677-683.
47. Morrison, S. A., Lorenz, D., Eskay, C. P., Forrest, G. F., & Basso, D. M. (2018). Longitudinal recovery and reduced costs after 120 sessions of locomotor training for motor incomplete spinal cord injury. Arch Phys Med Rehabil, 99(3), 555-562.
48. Nathan, B. P., Nisar, R., Short, J., Randall, S., Grissom, E., Griffin, G., Switzer, P. V., & Struble, R. G. (2005). Delayed olfactory nerve regeneration in ApoE-deficient mice. Brain Res, 1041(1), 87-94.
49. Nathan, P. W. (1994). Effects on movement of surgical incisions into the human spinal cord. Brain, 117, 337-346.
50. Norden, D. M., Faw, T. D., McKim, D. B., Deibert, R. J., Fisher, L. C., Sheridan, J. F., Godbout, J. P., & Basso, D. M. (2019). Bone Marrow-Derived Monocytes Drive the Inflammatory Microenvironment in Local and Remote Regions after Thoracic Spinal Cord Injury. J Neurotrauma, 36(6), 937-949.
51. NSCISC. (2019, 2019). National Spinal Cord Injury Statisitcal Center. Spinal Cord Injury Facts and Figures at a Glance. University of Alabama.
52. Pan, W., & Kastin, A. J. (2008). Cytokine transport across the injured blood-spinal cord barrier. Curr Pharm Des, 14(16), 1620-1624.
53. Pocivavsek, A., Mikhailenko, I., Strickland, D. K., & Rebeck, G. W. (2009). Microglial low-density lipoprotein receptor-related protein 1 modulates c-Jun N-terminal kinase activation. J Neuroimmunol, 214(1-2), 25-32.
54. Popovich, P. G., Guan, Z., McGaughy, V., Fisher, L., Hickey, W. F., & Basso, D. M. (2002). The neuropathological and behavioral consequences of intraspinal microglial/macrophage activation. Journal of Neuropathology and Experimental Neurology, 61(7), 623-633.
55. Popovich, P. G., Guan, Z., Wei, P., Huitinga, I., van Rooijen, N., & Stokes, B. T. (1999).

Depletion of hematogenous macrophages promotes partial hindlimb recovery and neuroanatomical repair after experimental spinal cord injury. Experimental Neurology, 158(2), 351-365.
56. Popovich, P. G., & Hickey, W. F. (2001). Bone marrow chimeric rats reveal the unique distribution of resident and recruited macrophages in the contused rat spinal cord. Journal of Neuropathology and Experimental Neurology, 60(7), 676-685.
57. Popovich, P. G., Horner, P. J., Mullin, B. B., & Stokes, B. T. (1996). A quantitative spatial analysis of the blood spinal cord barrier. I. Permeability changes after experimental spinal contusion injury. Experimental Neurology, 142(2), 258-275.
58. Popovich, P. G., Wei, P., & Stokes, B. T. (1997). Cellular inflammatory response after spinal cord injury in Sprague-Dawley and Lewis rats. Journal of Comparative Neurology, 377(3), 443-464.
59. Prockop, L. D., Naidu, K. A., Binard, J. E., & Ransohoff, J. (1995). Selective permeability of [3H]-D-mannitol and [14C]-carboxyl-inulin across the blood-brain barrier and blood-spinal cord barrier in the rabbit. The journal of spinal cord medicine, 18(4), 221-226.
60. Rice, T., Larsen, J., Rivest, S., & Yong, V. W. (2007). Characterization of the Early Neuroinflammation After Spinal Cord Injury in Mice. Journal of Neuropathology & Experimental Neurology, 66(3), 184-195.
61. Schnell, L., Fearn, S., Klassen, H., Schwab, M. E., & Perry, V. H. (1999). Acute inflammatory responses to mechanical lesions in the CNS: differences between brain and spinal cord. European Journal of Neuroscience, 11(10), 3648-3658.
62. Seitz, A., Kragol, M., Aglow, E., Showe, L., & Heber-Katz, E. (2003). Apolipoprotein E expression after spinal cord injury in the mouse. Journal of neuroscience research, 71(3), 417-426.
63. Sheng, H., Laskowitz, D. T., Mackensen, G. B., Kudo, M., Pearlstein, R. D., & Warner, D. S. (1999). Apolipoprotein E deficiency worsens outcome from global cerebral ischemia in the mouse. Stroke, 30(5), 1118-1124.
64. Sun, C., Ji, G., Liu, Q., & Yao, M. (2011). Apolipoprotein E epsilon 4 allele and outcomes of traumatic spinal cord injury in a Chinese Han population. Mol Biol Rep, 38(7), 4793-4796.
65. Town, T., Nikolic, V., & Tan, J. (2005). The microglial “activation” continuum: from innate to adaptive responses. Journal of Neuroinflammation, 2(1), 24.
66. Tu, T. M., Kolls, B. J., Soderblom, E. J., Cantillana, V., Ferrell, P. D., Moseley, M. A., Wang, H., Dawson, H. N., & Laskowitz, D. T. (2017). Apolipoprotein E mimetic peptide, CN-105, improves outcomes in ischemic stroke. Ann Clin Transl Neurol, 4(4), 246-265.
67. Wang, H., Anderson, L. G., Lascola, C. D., James, M. L., Venkatraman, T. N., Bennett, E. R., Acheson, S. K., Vitek, M. P., & Laskowitz, D. T. (2013). ApolipoproteinE mimetic peptides improve outcome after focal ischemia. Exp Neurol, 241, 67-74.
68. Wang, R., Hong, J., Lu, M., Neil, J. E., Vitek, M. P., Liu, X., Warner, D. S., Li, F., & Sheng, H. (2014). ApoE mimetic ameliorates motor deficit and tissue damage in rat spinal cord injury. Journal of neuroscience research, 92(7), 884-892.
69. Weisgraber, K. H. (1994). Apolipoprotein E: Structure-Function Relationships. In C. B.

Anfinsen, J. T. Edsall, F. M. Richards, & D. S. Eisenberg (Eds.), Advances in Protein Chemistry (Vol. 45, pp. 249-302). Academic Press.
70. Winkler, E. A., Sengillo, J. D., Bell, R. D., Wang, J., & Zlokovic, B. V. (2012). Blood-spinal cord barrier pericyte reductions contribute to increased capillary permeability. J Cereb Blood Flow Metab, 32(10), 1841-1852.
71. Xue, M., Li, S., Xu, M., Yan, L., Laskowitz, D. T., Kolls, B. J., Chen, G., Qian, X., Wang, Y., Song, H., & Wang, Y. (2022). Antagonism of nicotinic acetycholinergic receptors by CN-105, an apoE-mimetic peptide reduces stroke-induced excitotoxicity. Clin Transl Med, 12(1), e677.
72. Yang, L., Liu, C.-C., Zheng, H., Kanekiyo, T., Atagi, Y., Jia, L., Wang, D., N'songo, A., Can, D., Xu, H., Chen, X.-F., & Bu, G. (2016). LRP1 modulates the microglial immune response via regulation of JNK and NF-xB signaling pathways. Journal of Neuroinflammation, 13(1), 304.
73. Yang, X., Chen, S., Shao, Z., Li, Y., Wu, H., Li, X., Mao, L., Zhou, Z., Bai, L., Mei, X., & Liu, C. (2018). Apolipoprotein E Deficiency Exacerbates Spinal Cord Injury in Mice: Inflammatory Response and Oxidative Stress Mediated by NF-kappaB Signaling Pathway. Front Cell Neurosci, 12, 142.
74. Zhou, W., Xu, D., Peng, X., Zhang, Q., Jia, J., & Crutcher, K. A. (2008). Meta-analysis of APOE4 allele and outcome after traumatic brain injury. J Neurotrauma, 25(4), 279-290.
75. Zhu, Y., Soderblom, C., Krishnan, V., Ashbaugh, J., Bethea, J. R., & Lee, J. K. (2015). Hematogenous macrophage depletion reduces the fibrotic scar and increases axonal growth after spinal cord injury. Neurobiol Dis, 74, 114-125.
76. Zurhove, K., Nakajima, C., Herz, J., Bock, H. H., & May, P. (2008). Gamma-secretase limits the inflammatory response through the processing of LRP1. Science signaling, 1(47), ra15-ra15.

The foregoing is illustrative of the present invention, and is not to be construed as limiting thereof. The invention is defined by the following claims, with equivalents of the claims to be included therein.

What is claimed is:

1. A method for improving a functional outcome of a spinal cord injury in a subject in need thereof, comprising administering to said subject an effective amount a peptide of Formula I:
X1-X2-X3-X4-X5 (SEQ ID NO:1)
or a salt thereof, wherein
X1 is V or R;
X2 is S, A, or H;
X3 is K or R;
X4 is K or R; and
X5 is R, L, or K,
or a pharmaceutically acceptable salt thereof,
optionally wherein said peptide is N-terminal acetylated and/or C-terminal amidated.

2. The method of claim 1, wherein the peptide of Formula I is: VSRKR (SEQ ID NO:2), VSKRR (SEQ ID NO:3), VSRRR (SEQ ID NO:4), VARKL (SEQ ID NO:5), RHKKL (SEQ ID NO:6), RARRL (SEQ ID NO:7), RSKKL (SEQ ID NO:8), RHKRR (SEQ ID NO:9), VARRL (SEQ ID NO:10), VARRK (SEQ ID NO:11), or RSKRR (SEQ ID NO:12), or a pharmaceutically acceptable salt thereof.

3. The method of claim 1, wherein the peptide of Formula I is selected from the group consisting of: 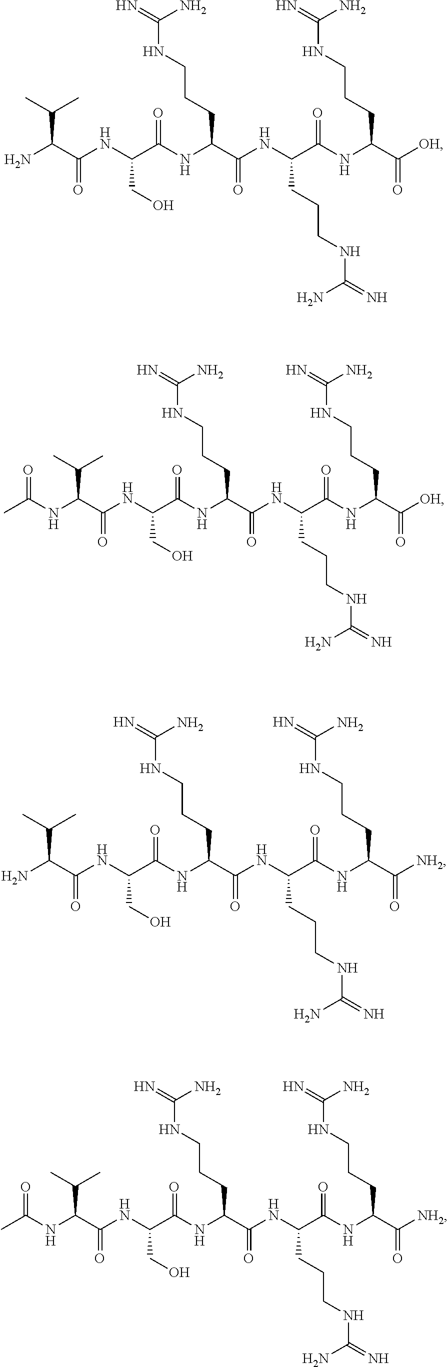 [SMILES: CC(C)[C@H](N)C(=O)N[C@@H](CO)C(=O)N[C@@H](CCCNC(=N)N)C(=O)N[C@@H](CCCNC(=N)N)C(=O)N[C@@H](CCCNC(=N)N)C(=O)O ; CC(=O)N[C@H](C(=O)N[C@@H](CO)C(=O)N[C@@H](CCCNC(=N)N)C(=O)N[C@@H](CCCNC(=N)N)C(=O)N[C@@H](CCCNC(=N)N)C(=O)O)C(C)C ; CC(C)[C@H](N)C(=O)N[C@@H](CO)C(=O)N[C@@H](CCCNC(=N)N)C(=O)N[C@@H](CCCNC(=N)N)C(=O)N[C@@H](CCCNC(=N)N)C(N)=O ; CC(=O)N[C@H](C(=O)N[C@@H](CO)C(=O)N[C@@H](CCCNC(=N)N)C(=O)N[C@@H](CCCNC(=N)N)C(=O)N[C@@H](CCCNC(=N)N)C(N)=O)C(C)C] 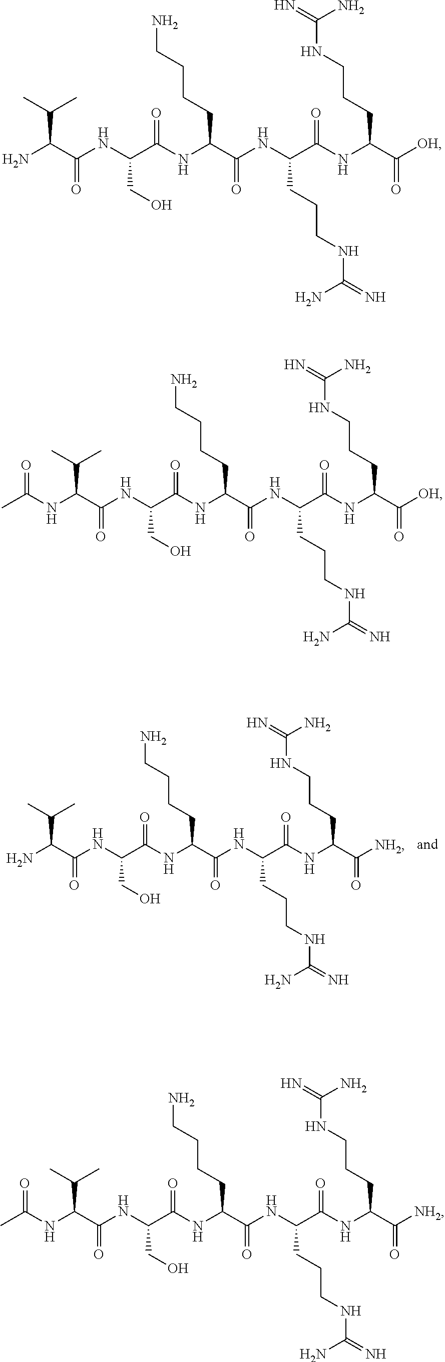 [SMILES: CC(C)[C@H](N)C(=O)N[C@@H](CO)C(=O)N[C@@H](CCCCN)C(=O)N[C@@H](CCCNC(=N)N)C(=O)N[C@@H](CCCNC(=N)N)C(=O)O ; CC(=O)N[C@H](C(=O)N[C@@H](CO)C(=O)N[C@@H](CCCCN)C(=O)N[C@@H](CCCNC(=N)N)C(=O)N[C@@H](CCCNC(=N)N)C(=O)O)C(C)C ; CC(C)[C@H](N)C(=O)N[C@@H](CO)C(=O)N[C@@H](CCCCN)C(=O)N[C@@H](CCCNC(=N)N)C(=O)N[C@@H](CCCNC(=N)N)C(N)=O ; CC(=O)N[C@H](C(=O)N[C@@H](CO)C(=O)N[C@@H](CCCCN)C(=O)N[C@@H](CCCNC(=N)N)C(=O)N[C@@H](CCCNC(=N)N)C(N)=O)C(C)C]
and pharmaceutically acceptable salts thereof.

4. The method of claim 1, wherein the peptide of Formula I is: 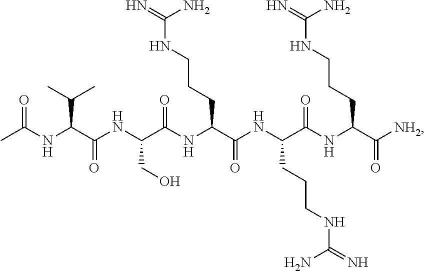 [SMILES: CC(=O)N[C@H](C(=O)N[C@@H](CO)C(=O)N[C@@H](CCCNC(=N)N)C(=O)N[C@@H](CCCNC(=N)N)C(=O)N[C@@H](CCCNC(=N)N)C(N)=O)C(C)C]
or a pharmaceutically acceptable salt thereof.

5. The method of claim 1, wherein the peptide of Formula I is: 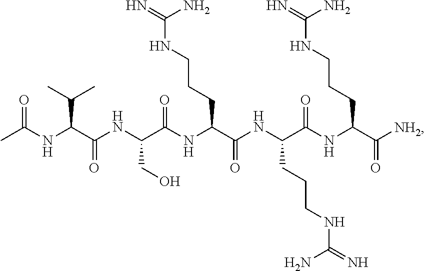 [SMILES: CC(=O)N[C@H](C(=O)N[C@@H](CO)C(=O)N[C@@H](CCCNC(=N)N)C(=O)N[C@@H](CCCNC(=N)N)C(=O)N[C@@H](CCCNC(=N)N)C(N)=O)C(C)C]
provided as an acetate salt.

6. The method of claim 1, wherein said peptide is provided in a pharmaceutically acceptable carrier.

7. The method of claim 6, wherein said pharmaceutically acceptable carrier is suitable for administering said peptide by injection or infusion.

8. The method of claim 1, wherein said spinal cord injury is motor and/or sensory complete.

9. The method of claim 1, wherein said spinal cord injury is motor and/or sensory incomplete.

10. The method of claim 1, wherein the functional outcome comprises sensory function.

11. The method of claim 1, wherein the functional outcome comprises motor function.

12. The method of claim 1, wherein the functional outcome comprises bladder function.

13. The method of claim 1, wherein said subject is a human subject.

14. The method of claim 1, wherein said administering is carried out by injection.

15. The method of claim 1, wherein said administering is carried out by intravenous administration.

16. The method of claim 1, wherein said administering is carried out by intraperitoneal administration.

17. The method of claim 1, wherein the peptide is administered to the subject at a dosage of from 0.01 to 10 mg/kg.

18. The method of claim 1, wherein the peptide is administered in the acute phase of the spinal cord injury.

19. The method of claim 1, wherein the peptide is administered in the subacute or chronic phase following the spinal cord injury.

20. The method of claim 1, wherein the peptide is administered in combination with a rehabilitative or engineering intervention for the spinal cord injury.
